(19) United States
(12) Patent Application Publication
(10) Pub. No.: US 2024/0156614 A1
(43) Pub. Date: May 16, 2024

(54) INTERBODY DEVICE

(71) Applicant: EMMEMME S.R.L.S., Roma (IT)

(72) Inventors: Antonino RACO, Roma (IT); Stefano CASELLI, Roma (IT); Massimo MISCUSI, Roma (IT); Claudio TRAVERSO, Roma (IT)

(21) Appl. No.: 18/282,496

(22) PCT Filed: Mar. 11, 2022
(86) PCT No.: PCT/EP2022/000026

§ 371 Date: Sep. 15, 2023

(30) Foreign Application Priority Data
Mar. 18, 2021 (IT) 102021000005699

(51) Int. Cl. A61F 2/44

(52) U.S. Cl. CPC A61F 2/4465; A61F 2002/30354

(57) ABSTRACT

The invention relates to an expandable interbody device used for interbody operations on the spinal column and belongs to the field of medical equipment.

### TECHNICAL FIELD

The invention relates to an expandable interbody device used for interbody operations on the spinal column and belongs to the field of medical equipment.

### BACKGROUND ART

Vertebral pathologies have degeneration of the intervertebral discs as their primary cause.

The diminished ability of an intervertebral disc to perform its functions often results in the need to intervene thereon and consequently the possibility of damage to the motion segment of the spinal column.

Among the surgical treatments to treat this degeneration, the leading method today is a procedure for vertebral stabilisation involving intervertebral arthrodesis, which is an operation to re-establish the right distance between two or more deteriorated intervertebral discs which are no longer able to perform their function.

Interbody stabilisation is nowadays achieved through different surgical methods according to the anatomical district concerned and the location of the pre-eminent cause of pain. The devices which are placed in the interbody space consist of cages made of different materials and having different shapes. The main drawback of the cages lies in the fact that they can only act as spacers or as spacers/containers of a substance designed to generate the biological stabilisation of the segment undergoing treatment.

It must also be said that, for some surgical procedures, devices are used that can increase the overall height thereof, as the cages are expandable.

### DISCLOSURE OF INVENTION

With the aim of optimising the contact area between the device and the host anatomical site, the invention concerns a device which, while maintaining anatomically and mechanically adequate conditions, can increase in volume inside the interbody space until the said device covers a greater area of the host vertebral endplates.

In addition, the expansion can take place within the transverse anatomical plane, a particularity currently not available in commonly known devices.

In greater detail, the expansion of the device can take place through the displacement of the components thereof, which can reciprocally slide and move away from one another so as to ensure greater coverage of the interbody space.

Thanks to the said characteristic of the invention, operators will be able to carry out, in a minimally invasive way, surgical procedures which are necessary in order to be able to position interbody devices which must cover a greater area of the vertebral endplates.

In addition to this particularity, the device allows extension along the coronal plane in order to possibly achieve better filling of the intervention space.

### BRIEF DESCRIPTION OF DRAWINGS

The characteristics of the invention in question will emerge more clearly in the following descriptions of preferred embodiments in accordance with the descriptions in the claims, with the aid of the annexed drawings, in which:

FIG. 1 shows the device in question in the present invention in a closed, i.e. compact, configuration;

FIG. 2 shows the device in question in the present invention in an expanded configuration;

FIG. 3 shows the device in the previous figures from a perspective view;

FIGS. 4 to 6 show only the structure of the device for a better view of the operation mechanism.

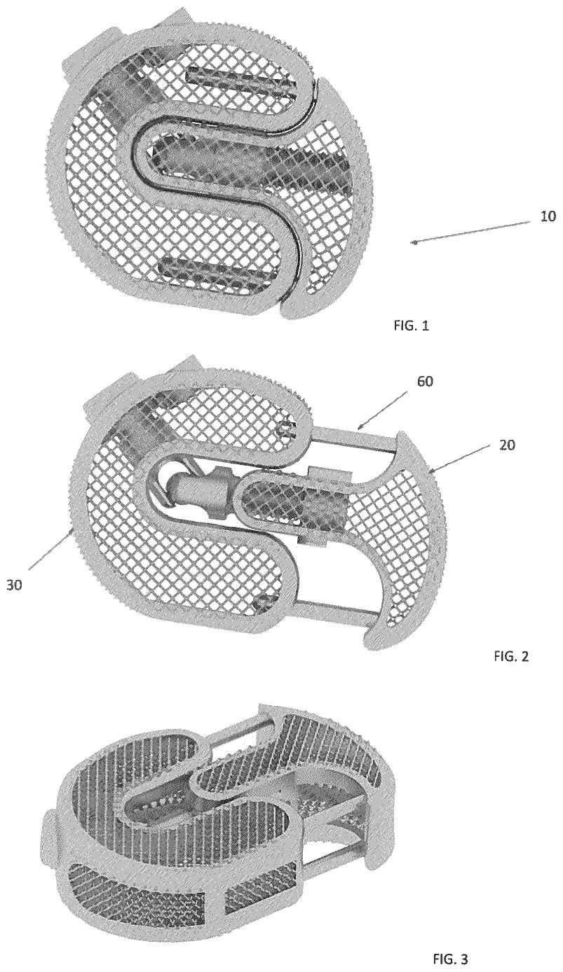

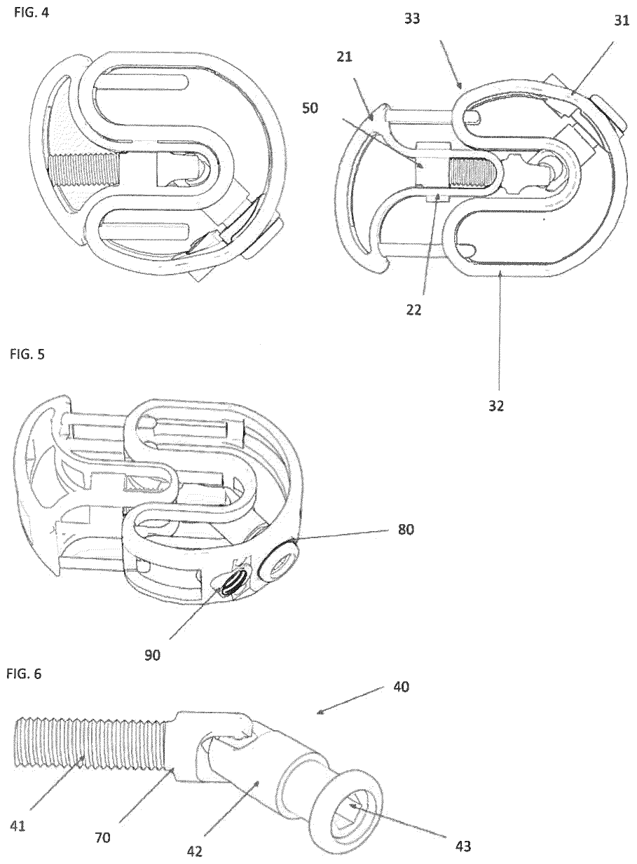

### BEST MODE FOR CARRYING OUT THE INVENTION

With reference to the figures shown, 10 denotes an expandable interbody device as a whole, the said device essentially consisting of two movable and mutually sliding elements 20 and 30; in this way, while maintaining anatomically and mechanically appropriate conditions, the device 10 may be adjusted so as to increase in volume in the interbody space until the said device covers a greater area of the host vertebral endplates.

For this purpose, the two elements 20 and 30 comprise respective base portions 21, 31 configured precisely to expand within the interbody space until the said elements cover a greater area of the vertebral endplates.

Coupled with each base portion 21, 32 are one or more extensions, hereinafter referred to as sliding teeth, configured precisely so as to slide in a mutual fashion during operation of the device 10.

More precisely, in the embodiment shown here, by way of example, the base portion 21 is equipped with a single tooth 22 which extends therefrom and may be housed, in at least one compact configuration of the device 10, between two corresponding teeth 32 and 33 on the portion 30; it remains understood, however, that this characterisation concerning the number of teeth must not be understood as limiting the protection sought and that other equivalent embodiments obvious to a person skilled in the art fall within the scope of protection of the present invention.

Essentially, the portions 20 and 30 are configured so as to move closer to and away from each other, also supported by the respective sliding teeth 22, 32, 33 and possibly by guides, as described below; thanks to this characteristic, the device 10 finds itself in two limit operating conditions, namely:
a first “compact” or closed configuration of the device 10, in which the first element 20 is almost in contact with the second element 30 and one or more teeth on the first element 20 are between those on the second element 30,
a second “extended” configuration, i.e. open and having a greater volume, in which the first element 20 is spaced apart from the second element 30, i.e. the teeth on the first element 20 are interfering with those of the second element 30 in a minimal fashion.

In this way, the interbody device 10 can change from the first to the second configuration, and vice versa; in this regard, the device 10 is coupled with an instrument so that, once positioned in the interbody space, the device 10 is expanded horizontally by means of a suitable dedicated instrument, which acts on a adjustment mechanism 40, described below, which generates the mutual spacing of the elements and therefore the opening of the device 10, so as to fill the entire interbody space.

It can be seen that the device 10 in a “compact” configuration, i.e. closed, has the ends of the teeth on an element almost in contact with, or at least in proximity to, the internal side of the base portion of the other element, to obtain the minimum overall dimensions of the said device.

As already mentioned, according to one advantageous aspect of the invention, the device 10 may comprise one or more guide pins 60, for better control and stabilisation in the “extended” configuration.

More specifically, in the example shown, the presence of four stabilisation pins 60 connected to the base portion 21 of the body 20, from which they extend in a linear fashion, in the same direction as the tooth 22, with the result that each pin 60 enters and is guided by respective holes obtained in the body 30, precisely in the end portions of the teeth 32 and 33.

The passage from the compact to the extended configuration (and vice versa) is carried out, as anticipated, by means of an adjustment mechanism 40 housed inside the device 10 configured to cooperate with the respective elements 20 and 30 by means of a female screw 50.

In greater detail, the adjustment mechanism 40 comprises a first threaded pin 41 which is connected to a second pin 42 by means of a transmission member such as a cardan 70, which transmits motion between the respective two misaligned axes of the pins 41 and 42, whose extensions are incident at a point.

The thread of the first pin 41 engages in the corresponding female screw 50 which is integral with one of the two elements of the device, while the second pin 42 is integral with the other element since the said pin is housed in a circular housing hole 80 which allows it to rotate on its own axis only.

Advantageously, the axes of the two pins 41 and 42 are arranged along the same plane which corresponds to the sliding plane of the elements 20 and 30, or the opening plane of the device 10.

The pin 41 and the female screw 50 are arranged so that the rotation axis corresponds with the opening/closing direction of the device 10.

The second pin 42, which is incident with respect to the first pin and inclined by a variable angle of approximately 45°, comprises a seat 43, with an anti-rotation geometry, formed inside the head portion; through the seat the device may be coupled with a control instrument (not shown) to allow easy opening/closing of the device 10.

According to one advantageous aspect of the invention, the opening mechanism 40 for opening the device 10 is made as a single piece and is not assembled but rather is made by means of titanium sintering already assembled with the movement calibrated.

The elements 20 and 30 of the device 10 are also made by sintering; the said elements consist of a structural skeleton that essentially follows the contours of the various elements, and which is “lightened” both on the inside thereof (as the elements are hollow) and on the surface due to the presence of a reticular texture which guarantees high mechanical strength (while using less material) and improved biomechanical stability of the interbody device.

The expanding interbody device 10 is operated by means of an implant holder, in other words a surgical instrument onto which it is fastened by means of the threaded fixing hole 90 therein.

Through a surgical procedure called OLIF (Oblique Lumbar Interbody Fusion) the device enters the lumbar interbody space.

Once the device 10 has been positioned inside the interbody space, the said device is expanded by means of the dedicated surgical instrument, which fastens into the hexagonal seat 43 next to the threaded fixing hole; through a clockwise rotation of the handpiece, the opening of the device 10 is generated and the said device thus fills the entire interbody space.

1. A device (10), in particular for intervertebral stabilisation, characterised by the fact that the said device comprises at least two bodies (20, 30) which slide mutually one into the other and cooperate so as to increase in volume in the intervertebral space until the said device covers a greater area of the host vertebral endplates; and in which an adjustment mechanism (40) interacts with the said bodies (20, 30) so as to determine:
a. a first “compact” or closed condition, in which the device (10) moves so that the teeth on the first element (20) are between those of the second element (30), or vice versa, and
b. a second “extended” configuration, i.e. open and having a greater volume, in which the teeth of the first element (20) are displaced and interfere in a minimal fashion with those of the second element (30).

2. A device (10) according to claim 1 wherein the adjustment mechanism (40)
a. is housed inside the device (10) and is configured to cooperate with the respective elements (20) and (30) by means of a female screw (50) which is integral with one of said elements (20, 30);
b. comprises a first pin (41) and a second pin (42) connected to each other by means of a transmission member such as a cardan (70), which transmits the motion between the non-aligned axes of the pins (41) and (42)
and in which the first pin (41) is endowed with a thread suitable for engaging with the corresponding female screw (50), and the second pin (42) is housed in a circular housing hole (80) that allows it to rotate on its own axis only.

3. A device (10) according to claim 1 wherein the axes of the two pins (41) and (42) are arranged along the same plane which corresponds to the sliding plane of the elements (20) and (30), i.e. the opening of the device (10) and the pin (41) and female screw (50) being arranged with an axis of rotation corresponding to the said opening direction.

4. A device (10) according to claim 1 wherein the second pin (42) comprises a seat (43) with an anti-rotation geometry formed inside the head portion.

5. A device (10) according to claim 1 wherein the opening mechanism 40 is made as a single piece and is not assembled but rather is made by means of titanium sintering already assembled with the movement calibrated.
